(19) United States
(12) Patent Application Publication
(10) Pub. No.: US 2008/0319283 A1
(43) Pub. Date: Dec. 25, 2008

(54) METHOD AND APPARATUS FOR MEASURING SKIN TEXTURE

(75) Inventors: Symon Cotton, Great Gransden (GB); Robert Morse, Cambridge (GB); Mark Chellingworth, Vale of Glamorgan (GB)

Correspondence Address: BROOKS, CAMERON & HUEBSCH , PLLC, 1221 NICOLLET AVENUE , SUITE 500, MINNEAPOLIS, MN, 55403, US

(73) Assignee: Astron Clinica Limited, Cambridge (GB)

(21) Appl. No.: 12/142,456

(22) Filed: Jun. 19, 2008

(30) Foreign Application Priority Data
Jun. 19, 2007 (EP) 07252483.8

(51) Int. Cl. A61B 5/00

(52) U.S. Cl. 600/306

(57) ABSTRACT

Among various methods, apparatuses, and media, a number of methods are provided for measuring skin surface texture. One such method includes illuminating an area of skin with polarized light, and obtaining a measurement of light returned by the illuminated area of skin in a first and a second waveband. The method includes processing the measurement of light in the first waveband to determine an estimated expected level of light in the second waveband returned by the illuminated area of skin utilising a model of the interaction of light with at least one chromophore in the skin. A measurement of the surface texture of the imaged illuminated area of skin can be determined on the basis of a difference between the estimated and actual levels of light in said second waveband returned by the illuminated area of skin.

### TECHNICAL FIELD

The present application relates to methods and apparatuses for measuring skin texture. In particular, embodiments of the present disclosure concern methods and apparatuses for measuring skin texture.

### BACKGROUND

When the skin is viewed in close up, the surface is composed of fine lines and wrinkles. Detailed measurements of these structures are of great interest in both the research of products designed to reduce the appearance of wrinkles and also in the education of consumers. In some instances, techniques to measure the topology of skin range from making physical silicon replicas of the skin, which are then traced, to stereo and fringe projection. Such techniques may produce useful results, but may require laboratory analysis that is limited due to costs and acquisition times.

### BRIEF DESCRIPTION OF THE DRAWINGS

FIG. 1 is a schematic cross sectional view through a layer of skin illustrating the structure of the skin and the interaction of that structure with incident light.

FIG. 2 is a schematic block diagram illustrating a skin texture measurement system in accordance with at least one embodiment of the present disclosure.

FIG. 3 is a flow diagram illustrating the processing performed by the skin texture measurement system of FIG. 2 in accordance with at least one embodiment of the present disclosure.

FIG. 4 is a graph illustrating the relationship between the reflection of red and infra-red light by skin with a fixed amount of collagen.

### DETAILED DESCRIPTION OF THE DISCLOSURE

Among various methods, apparatuses, and media, a number of methods are provided for measuring skin surface texture. One such method includes illuminating an area of skin with polarized light, and obtaining a measurement of light returned by the illuminated area of skin in a first and a second waveband. The method includes processing the measurement of light in the first waveband to determine an estimated expected level of light in the second waveband returned by the illuminated area of skin utilising a model of the interaction of light with at least one chromophore in the skin. A measurement of the surface texture of the imaged illuminated area of skin can be determined on the basis of a difference between the estimated and actual levels of light in the second waveband returned by the illuminated area of skin.

In various embodiments, such a method can include obtaining the measurement of light returned by the illuminated area of skin in the first and the second waveband, where the measured light in the first waveband is light having a different polarity to the light with which the area of skin is illuminated and the measured light in the second waveband includes light having the same and different polarities of light as the light with which the area of skin is illuminated.

The present disclosure also provides, in various embodiments, apparatuses for measuring skin surface texture, where the apparatuses include a light source operable to illuminate an area of skin with polarized light. Such apparatuses can, in various embodiments, include a detector operable to obtain a measurement of light returned by an illuminated area of skin in a first waveband and a second waveband, and a processor operable to process an obtained measurement of light in a first waveband to determine an estimated expected level of light in a second waveband returned by an illuminated area of skin utilising a model of the interaction of light with at least one chromophore in the skin. The processor can determine a measurement of the surface texture of an imaged illuminated area of skin on the basis of a difference between estimated and obtained actual levels of light in the second waveband returned by an illuminated area of skin.

In various embodiments, such apparatuses can include the detector operable to obtain the measurement of light returned the illuminated area of skin in the first waveband and the second waveband, where the measured light in the first waveband is light having a different polarity to the light with which the area of skin is illuminated by the light source and the measured light in the second waveband includes light having the same and different polarities of light as the light with which the area of skin is illuminated by the light source.

The present disclosure further provides, in various embodiments, a recording medium storing instructions for causing execution of such instructions in order to receive an obtained measurement of light returned by an illuminated area of skin in a first and a second waveband, where the measured light in the first waveband is light having a different polarity to the light with which the area of skin is illuminated and the measured light in the second waveband includes light having the same and different polarities of light as the light with which the area of skin is illuminated. Such instructions, in various embodiments, can be executed to process a received measurement of light in the first waveband to determine an estimated expected level of light in the second waveband returned by the illuminated area of skin utilising a model of the interaction of light with at least one chromophore in the skin. Execution of such instructions can determine a measurement of a surface texture of the imaged illuminated area of skin on the basis of a difference between estimated and actual levels of light in the second waveband returned by the illuminated area of skin.

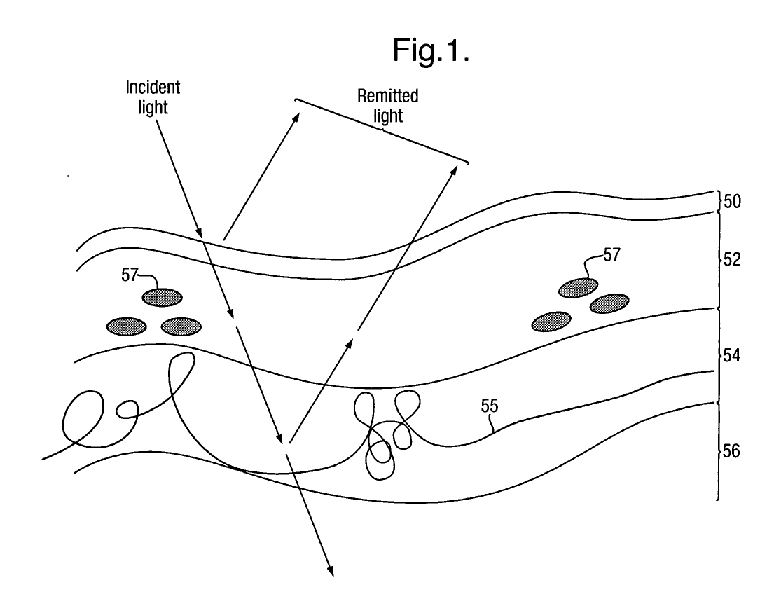

FIG. 1 is a schematic cross sectional view through a layer of skin illustrating the structure of the skin and the interaction of that structure with incident light. To assist understanding, the physical structure of skin and the interaction of skin with light will first be briefly explained with reference to FIG. 1.

As shown in FIG. 1, skin has a layered structure including an outer cornified layer 50 also known as the stratum corneum, the epidermis 52, and the dermis which itself can be divided into the papillary dermis 54 which contains the blood supply 55 for the skin and the reticular dermis 56.

When light is incident on the skin, much of the light is immediately reflected when coming into contact with the outer cornified layer 50. A proportion of incident light does, however, pass through the cornified layer 50 and proceeds to interact with the constituents of the epidermis 52 and the papillary dermis 54. As light passes through the epidermis 52 and the papillary dermis 54 the light is absorbed by various chromophores present in the skin, most notably chromophores such as haemoglobin present in the blood in blood vessels 55 in the papillary dermis, melanin, a pigment produced by melanocytes 57 in the epidermis 52 and collagen a fibrous material present throughout the skin. By the time the incident light reaches the reticular dermis 56 the scattering of light is highly forward and therefore for that reason the reticular dermis 56 can for all intents and purposes be considered returning no light.

In addition to chromophores present in the epidermis 52 and papillary dermis 54 absorbing various wavelengths, certain structures in the skin most notably collagen cause incident light to be reflected. The outward appearance of the skin can therefore be considered to be a mixture of the light immediately reflected by the cornified layer 50 and the remitted light which has interacted with the chromophores present in the epidermis 52 and the papillary dermis 54.

As will be described, the present disclosure utilises the fact that the appearance of the skin is dependent upon the reflection of light from the surface of the skin and the interaction of light with structures and chromophores below the surface to obtain a measurement of the skin's surface texture.

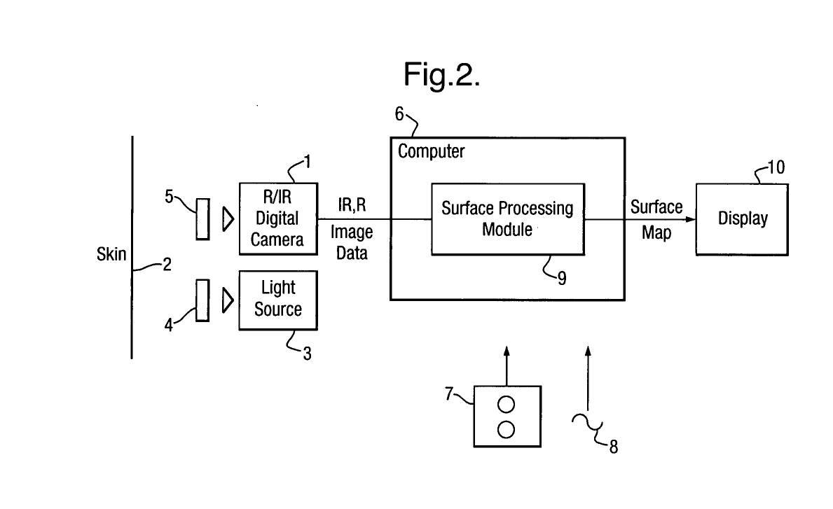

FIG. 2 is a schematic block diagram illustrating a skin texture measurement system in accordance with at least one embodiment of the present disclosure. Referring to FIG. 2, which is a schematic block diagram of an embodiment of the present disclosure, a digital camera 1 including a digital camera operable to obtain red and infra-red images of light with wavelengths of approximately 650 nm and 900 nm respectively is provided which is arranged to obtain an image of the surface of the skin of an individual 2 illuminated by a light source 3.

Provided in front of the lens of the digital camera 1 and the light source 3 are a first 4 and a second polarizer 5. These polarizers 4, 5 are conventional polarizers which polarize visible light having wavelengths in the range of 400 to 700 nanometers (nm) with the second polarizer 5 being arranged so as to be cross polarized with the first 3.

The interaction of light with collagen in the skin is such to cause the light to loose its original polarization. Light detected by the red detectors of the digital camera 1 when an area of skin 2 is illumined by the light source 3 via the first polarizer 4 therefore includes red light which has passed through the surface of the skin and interacted with the chromophores and collagen in the skin below the surface. This is because the polarized red light directly reflected from the surface of the skin will be filtered by the cross polarization of the second polarizer 5 in front of the lens of the digital camera 1.

In contrast, light detected by the infra-red detectors of the digital camera 1 when an area of skin 2 is illuminated by the light source 3 via the first polarizer 4 will pass through the second polarizer 5 regardless of whether the light has had its polarization altered through interaction with collagen in the skin since the range of the polarizers 4, 5 does not extend to infra-red light. The infra-red light detected by the digital camera 1 will therefore include a mixture of infra-red light which has been reflected directly from the surface of the skin 2 infra-red light which has interacted with the chromophores and structures of the skin 2 below the surface.

The red and infra-red images obtained by the digital camera 1 are then transmitted to a computer 6 which is configured by software either provided on a disk 7 or by receiving an electrical signal 8 by via a communications network to be configured to include a surface processing module 9 to process the image data in the manner described below to generate a surface map illustrating the detailed variations in the surface of the skin 2 imaged by the camera 1. This surface map is then shown on a display 10.

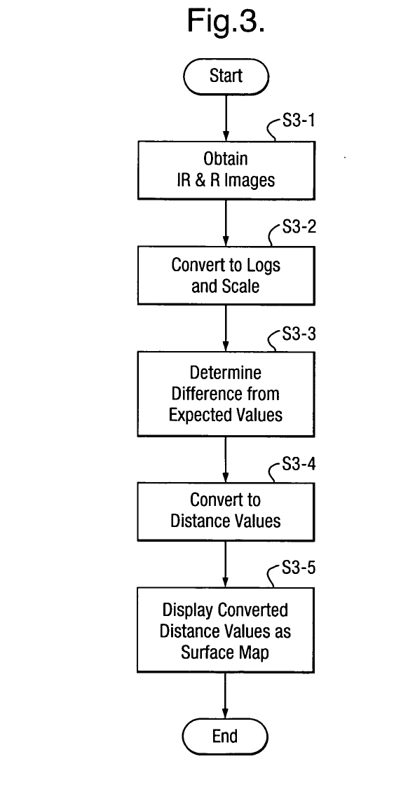

FIG. 3 is a flow diagram illustrating the processing performed by the skin texture measurement system of FIG. 2 in accordance with at least one embodiment of the present disclosure. Referring to FIG. 3, which is a flow diagram of the processing performed by the computer 6 of FIG. 2, initially (S3-1) an image is obtained by the digital camera 1 of the area of skin 2 illuminated by the light source 3.

In this embodiment image data generated by the digital camera 1 includes R and IR values ranging from 0 to 255 for a large array of pixels where the R and IR values are indicative of the extent light received by a photo receptor within the camera 1 for each pixel in an image appears to be red or infra-red where a completely cold black pixel has R and IR values of 0, 0 and a completely hot bright white pixel has R and IR values of 255, 255.

When an image of an area of skin 2 has been obtained by the camera 1, the surface processing module 9 then proceeds to process (S3-2-S3-4) each pair of R, IR pixel values in the obtained image in turn to convert the R, IR pixel values into values indicative of surface texture.

In this embodiment, this conversion is based upon two assumptions.

Firstly, it is assumed that the skin surface 2 is substantially flat and the illumination of the skin surface is substantially uniform. This will be the case where a small area of skin in being imaged and it is possible to bring the light source 3 and camera 1 into close proximity of the skin 2 being analysed.

Secondly, it is assumed that the area of skin is a healthy area of skin with uniform a thickness of collagen of 0.2 millimeter (mm).

Under such circumstance, the ratio of the red and infra-red light detected can be considered as only affected by variations in concentrations of melanin and small scale variations in the surface of the skin the since both red and infra-red light is substantially unaffected by the presence of haemoglobin.

In this embodiment, natural logarithms of the R and IR values for a pixel are first taken and then the resultant logarithms are scaled so as to fall been a minimum value of 0 and a maximum value of 1 (S3-2). The difference between the actual scaled logarithm of the detected infra-red value IR is then compared (S3-3) with an expected infra-red value derived from the scaled logarithm of the detected red value R.

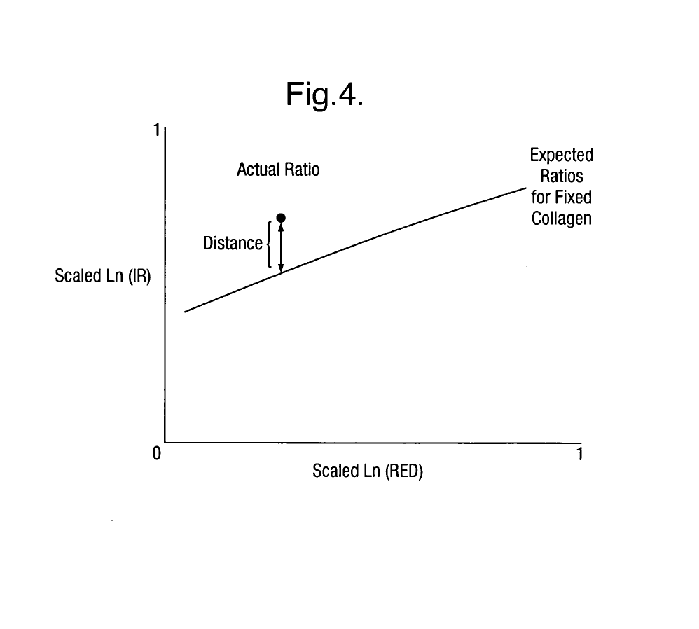

FIG. 4 is a graph illustrating the relationship between the reflection of red and infra-red light by skin with a fixed amount of collagen. By way of example and not by way of limitation, FIG. 4 illustrates the relationship between the reflection of red and infra-red light by skin with a fixed amount of collagen in the absence of any surface reflection. In such circumstance the ratio of light is entirely dependent upon the concentration of melanin present within the epidermis which can be considered to be a perfect exponential term. In the graph of graph of FIG. 4 where the axes are scaled logarithmic axes, this means that expected ratios of red and infra-red values fall on a straight line. The difference between an expected infra-red value and the actual value derived by scaling the logarithm of the IR value for a pixel arises due to the occurrence of surface reflection. A measurement of the surface texture at a point corresponding to a pixel in an obtained image can then be obtained (S3-4) by taking the antilog of the calculated distance between the actual infra-red value and the expected infra-red value determined from the detected level of reflected red light.

This process (S3-2-S3-4) is then repeated for all of the pixels in the obtained images and the resultant converted difference values are then displayed (S3-5) as a surface map.

In generating the surface map, although the assumption that the thickness of collagen is uniform is not likely to be true, variations in converted distance due to the usual variation in collagen thickness within the range of normal skin are significantly smaller than the variations arising due to variations arising to differences in surface reflection to differences in the surface topology of the skin and hence do not have an appreciable impact on the accuracy of the obtained measurements.

In the resultant surface map, pixels where little or non-surface reflection has occurred which will correspond to wrinkles or furrows in the skin will be associated with lower values with the relative size of the measurement indicative of the depth of the furrow or wrinkle. Additionally, the obtained map can also be used to measure the extent of areas of dry skin as such areas are associated with higher converted distance values and areas of surface maps indicative of more alpine skin topology.

Although in the above described embodiment a skin texture analysis system has been described which processes red and infrared images, alternative systems could be used.

Thus, for example, instead of a red/infra-red digital camera, a conventional RGB camera could be utilised. In such an alternative embodiment polarizers would have to be provided which did not extend through the entire range of detection of the camera so that at least one image could be obtained which was an image based on a mixture of light directly reflected from the surface of the skin and light which interacts with the structures and chromophores in the skin.

Although in the above embodiment a measure of skin texture is obtained using two images of the skin more images could be utilised. More specifically, in the above embodiment red and infra-red images are processed to obtain a skin surface measurement. Utilising red and infra-red images is preferable because light of these wavelengths is substantially unaffected by the presence of haemoglobin. In other embodiments an additional color image, for example one based on green light could be obtained. The detected levels of green and red light could then be utilised to determine estimates of both blood and melanin concentrations present in the skin. The expected levels of infra-red light based on the determined concentrations could then be compared with the actual detected levels to determine a measurement of surface texture.

Although the embodiments of the disclosure described with reference to the drawings include computer apparatus and processes performed in computer apparatus, the disclosure also extends to computer programs, particularly computer programs on or in a carrier, adapted for putting the disclosure into practice. The program may be in the form of source or object code or in any other form suitable for use in the implementation of the processes according to the disclosure. Additionally, the carrier can be any entity or device capable of carrying and/or executing the program, such as various types of individual or interacting software, firmware, hardware, Flash drives, logic, and application-specific integrated circuits, among others, installed in one or more locations.

For example, the carrier may include a storage medium, such as a ROM, for example a CD ROM or a semiconductor ROM, or a magnetic recording medium, for example a floppy disc or hard disk. Further, the carrier may be a transmissible carrier such as an electrical or optical signal which may be conveyed via electrical or optical cable or by radio or other means.

When a program is embodied in a signal which may be conveyed directly by a cable or other device or means, the carrier may be constituted by such cable or other device or means.

Alternatively, the carrier may be an integrated circuit in which the program is embedded, the integrated circuit being adapted for performing, or for use in the performance of, the relevant processes.

Although specific embodiments have been illustrated and described herein, those of ordinary skill in the relevant art will appreciate that an arrangement calculated to achieve the same results can be substituted for the specific embodiments shown. This disclosure is intended to cover all adaptations or variations of various embodiments of the present disclosure.

Reference is made to various specific embodiments in which the disclosure may be practiced herein. These embodiments are described with sufficient detail to enable those skilled in the art to practice the disclosure. It is to be understood, however, that changes may be implemented to structural, logical, and electrical components to achieve the same results and still remain within the teachings of the present disclosure.

It is to be further understood that the above description has been made in an illustrative fashion, and not a restrictive one. Combination of the above embodiments, and other embodiments not specifically described herein, will be apparent to those of ordinary skill in the relevant art upon reviewing the above description.

The applicability of the various embodiments of the present disclosure includes other applications in which the above structures, devices, systems, and methods are used, for example, in implementations other than computer systems. Therefore, the applicability of various embodiments of the present disclosure should be determined with reference to the appended claims, along with the full range of equivalents to which such claims are entitled.

In the foregoing Detailed Description, various features are grouped together in a single embodiment for the purpose of streamlining the disclosure. This method of disclosure is not to be interpreted as reflecting an intention that the disclosed embodiments of the present disclosure need to use more features than are expressly recited in each claim Rather, as the following claims reflect, inventive subject matter lies in less than all features of a single disclosed embodiment. Thus, the following claims are hereby incorporated into the Detailed Description, with each claim standing on its own as a separate embodiment.

1. A method of measuring skin surface texture comprising:
illuminating an area of skin with polarized light;
obtaining a measurement of light returned by the illuminated area of skin in a first and a second waveband, wherein the measured light in the first waveband is light having a different polarity to the light with which said area of skin is illuminated and the measured light in the second waveband comprises light having the same and different polarities of light as the light with which said area of skin is illuminated;
processing the measurement of light in the first waveband to determine an estimated expected level of light in said second waveband returned by the illuminated area of skin utilising a model of the interaction of light with at least one chromophore in the skin; and
determining a measurement of the surface texture of the imaged illuminated area of skin on the basis of a difference between the estimated and actual levels of light in said second waveband returned by the illuminated area of skin.

2. The method of claim 1 wherein said first waveband comprises a waveband corresponding to visible light.

3. The method of claim 2 wherein said first waveband comprises a waveband corresponding to red light.

4. The method of claim 1 wherein said second waveband comprises a waveband corresponding to infra-red light.

5. The method of claim 1 wherein said at least one chromophore in the skin comprises melanin.

6. The method of claim 1, further comprising:
obtaining a measurement of light returned by the illuminated area of skin in a third waveband, wherein the measured light in the third waveband is light having a different polarity to the light with which said area of skin is illuminated;
wherein processing the first measurement of light to determine an estimated level of light in said second waveband returned by the illuminated area of skin comprises:
processing the measurements of light in said first and third wavebands to determine an estimated expected level of light in said second waveband returned by the illuminated area of skin utilising a model of the interaction of light with a first and a second chromophore in the skin.

7. The method of claim 6, wherein said a first and a second chromophore comprise melanin and haemoglobin.

8. An apparatus for measuring skin surface texture, the apparatus comprising:
a light source operable to illuminate an area of skin with polarized light;
a detector operable to:
obtain a measurement of light returned by an illuminated area of skin in a first waveband and a second waveband, wherein the measured light in the first waveband is light having a different polarity to the light with which said area of skin is illuminated by said light source, and the measured light in the second waveband comprises light having the same and different polarities of light as the light with which said area of skin is illuminated by said light source; and
a processor operable to:
process an obtained measurement of light in a first waveband to determine an estimated expected level of light in a second waveband returned by an illuminated area of skin utilising a model of the interaction of light with at least one chromophore in the skin; and
determine a measurement of the surface texture of an imaged illuminated area of skin on the basis of the difference between estimated and obtained actual levels of light in said second waveband returned by an illuminated area of skin.

9. The apparatus of claim 8 wherein said light source operable to illuminate an area of skin with polarized light comprises a light source operable to illuminate an area of skin via a polarizing filter.

10. The apparatus of claim 8 wherein said detector comprises:
a digital camera operable to obtain an image of an illuminated area of skin via a polarizing filter wherein the polarizing filter is operable to polarize light in said first waveband without polarizing light in said second waveband.

11. The apparatus of claim 8 wherein said first waveband comprises a waveband corresponding to visible light.

12. The apparatus of claim 11 wherein said first waveband comprises a waveband corresponding to red light.

13. The apparatus of claim 8 wherein said second waveband comprises a waveband corresponding to infra-red light.

14. The apparatus of claim 8, wherein said processor operable to:
process an obtained measurement of light in a first waveband to determine an expected value of light in said second waveband returned by the illuminated area of skin utilising a model of the interaction of light with melanin in the skin.

15. The apparatus of claims 7, wherein said detector is operable to obtain a measurement of light in a third waveband returned by an illuminated area of skin, wherein the measured light in the third waveband is light having a different polarity to the light with which said area of skin is illuminated; and
wherein said processor is operable to:
process obtained measurements of light in said first and third wavebands to determine an estimated expected level of light in said second waveband returned by an illuminated area of skin utilising a model of the interaction of light with a first and a second chromophore in the skin.

16. The apparatus of claim 15, wherein said a first and a second chromophore comprise melanin and haemoglobin.

17. A recording medium storing computer interpretable instructions for causing a programmable computer to be configured to execute such instructions in order to:
receive an obtained measurement of light returned by an illuminated area of skin in a first and a second waveband, wherein the measured light in the first waveband is light having a different polarity to the light with which said area of skin is illuminated and the measured light in the second waveband comprises light having the same and different polarities of light as the light with which said area of skin is illuminated;
process a received measurement of light in the first waveband to determine an estimated expected level of light in said second waveband returned by the illuminated area of skin utilising a model of the interaction of light with at least one chromophore in the skin; and
determine a measurement of a surface texture of the imaged illuminated area of skin on the basis of a difference between estimated and actual levels of light in said second waveband returned by the illuminated area of skin.

18. A recording medium in accordance with claim 17 wherein said at least one chromophore in the skin comprises melanin.

19. A recording medium in accordance with claim 17, further storing computer interpretable instructions for causing a programmable computer to be configured to execute such instructions in order to:
receive an obtained measurement of light returned by an illuminated area of skin in a third waveband, wherein the measured light in the third waveband is light having a different polarity to the light with which said area of skin is illuminated; and
process the measurements of light in said first and third wavebands to determine an estimated expected level of light in said second waveband returned by an illuminated area of skin utilising a model of the interaction of light with a first and a second chromophore in the skin.

20. A recording medium in accordance with claim 19, wherein said first and second chromophores comprise melanin and haemoglobin.

21. A recording medium in accordance with any of claims 17 comprising a computer disc.

22. A computer disc in accordance with claim 21 comprising a magnetic, optical or magneto-optical disc.
